(19) United States
(12) Patent Application Publication
(10) Pub. No.: US 2014/0236254 A1
(43) Pub. Date: Aug. 21, 2014

(54) DEVICES AND METHODS FOR TREATMENT OF THE ALIMENTARY TRACT

(71) Applicant: Boston Scientific Scimed, Inc., Maple Grove, MN (US)

(72) Inventors: Steve H. ZHANG, Blaine, MN (US); Kimberly A. MORRIS, Plymouth, MN (US); Pramodsingh H. THAKUR, Woodbury, MN (US); Sumit AGRAWAL, Plymouth, MN (US); David M. RAAB, Minneapolis, MN (US)

(73) Assignee: Boston Scientific Scimed, Inc., Maple Grove, MN (US)

(21) Appl. No.: 14/184,086

(22) Filed: Feb. 19, 2014

Related U.S. Application Data
(60) Provisional application No. 61/767,063, filed on Feb. 20, 2013.

(51) Int. Cl. A61N 1/36; A61F 5/00

(52) U.S. Cl. CPC A61N 1/36007; A61F 5/0026
USPC 607/40

(57) ABSTRACT

An alimentary tract treatment system is disclosed. The system may include a plurality of sensor devices for engaging a wall of a portion of the alimentary tract. The plurality of sensor devices may sense a parameter of the wall. The system may also include an alimentary tract treatment device for controlling a flow of material through the alimentary tract. Operation of the alimentary tract treatment device may be controlled based on the parameter sensed by the plurality of sensor devices.

### CROSS-REFERENCE TO RELATED APPLICATIONS

This application claims the benefit of priority from U.S. Provisional Application No. 61/767,063, filed on Feb. 20, 2013, the entirety of which is incorporated by reference herein.

### TECHNICAL FIELD

Various embodiments of the present disclosure relate generally to medical devices and related methods. More specifically, particular embodiments of the present disclosure relate to devices and methods for treating the alimentary tract, including, for example, treating gastroesophageal reflux disease (GERD) and obesity.

### BACKGROUND

GERD and obesity are conditions associated with the alimentary tract system. GERD is a condition in which stomach contents leak backwards from the stomach into the esophagus through the lower esophageal sphincter (LES). The leak may be caused by a weak LES or excess gastric pressure. The leaked contents may irritate the esophagus. The irritation may cause heartburn, and could also cause other symptoms. Obesity is a condition where a person's body stores an excess amount of fat, and can be caused by ingesting an excess amount of calories via the alimentary tract system.

Attempts have been made to manage GERD using laparoscopic devices and endoscopic devices. One type of laparoscopic device is a flexible band of interlinked beads with magnetic cores. The flexible band is positioned by laparoscopy around a portion of the esophagus surrounding the LES. The magnetic attraction between the interlinked beads helps keep the band in a contracted state, thus helping the LES resist opening. When food is swallowed, expansion of the esophagus forces the interlinked beads apart, temporarily breaking their magnetic bonds. After the food has passed, magnetic attraction between the interlinked beads is reestablished, thus assisting closing of the LES.

An alternative attempt at managing GERD involves placing a stent having a valve at the LES. The valve assists the LES by reducing or preventing unwanted backflow from the stomach into the esophagus. Another way of assisting closing of the LES involves electrically stimulating tissue at or around the LES to cause the LES to tighten.

Obesity may be managed by electrically stimulating a pyloric sphincter, causing the pyloric sphincter to tighten. Tightening of the pyloric sphincter can slow the rate at which the stomach empties of ingested contents. This may lead to a prolonged sensation of satiety, and a reduced desire for further caloric intake via the alimentary tract system.

Devices for GERD and obesity treatments should be able to differentiate between digestion events like swallowing, stomach contraction, and vomiting. However, current GERD and obesity treatment devices may be imprecise in the way they differentiate between digestion events. As such, the devices may not operate in the manner desired when a digestion event takes place.

In view of the above, the present methods and devices described herein provide improvements in treatments of the alimentary tract, including, for example, GERD and/or obesity treatments.

### SUMMARY

In accordance with certain embodiments of the present disclosure, an alimentary tract treatment system is disclosed. The system may include a plurality of sensor devices for engaging a wall of a portion of the alimentary tract. The plurality of sensor devices may also sense a parameter of the wall. The system may also include an alimentary tract treatment device for controlling a flow of material through the alimentary tract. Operation of the alimentary tract treatment device may be controlled based on the parameter sensed by the plurality of sensor devices.

In accordance with certain embodiments of the present disclosure, a method for treating gastroesophageal reflux disease (GERD) is disclosed. The method may include sensing peristaltic movement of a portion of an alimentary tract using a plurality of serially arranged strain sensor devices. The plurality of serially arranged strain sensor devices may be mounted on a wall of the alimentary tract. The plurality of serially arranged strain sensor devices may be operatively coupled to a GERD treatment device at a region of the alimentary tract including a lower esophageal sphincter (LES). The method may also include controlling operation of the GERD treatment device based on the sensed peristaltic movement.

In accordance with certain embodiments of the present disclosure, a method for treating obesity is disclosed. The method may include sensing a peristaltic movement of a portion of an alimentary tract using a plurality of serially arranged sensor devices. The plurality of serially arranged strain sensor devices may be mounted on a wall of the alimentary tract. The plurality of serially arranged strain sensor devices may be operatively coupled to an obesity treatment device at a pyloric region of the alimentary tract. The method may also include controlling operation of the obesity treatment device based on the sensed peristaltic movement.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is an illustration of a section of an alimentary tract including exemplary laparoscopic sensor assemblies, and an exemplary laparoscopic valve device, consistent with embodiments of the present disclosure;

FIG. 2 is an illustration of a cross-section of a section of the alimentary tract including exemplary endoscopic sensor assemblies, an exemplary endoscopic valve device, and exemplary electric stimulation devices, consistent with embodiments of the present disclosure;

FIG. 3 is a flow diagram of an exemplary method for GERD treatment, consistent with embodiments of the present disclosure;

FIG. 4 is a flow diagram of an exemplary method for GERD treatment, consistent with embodiments of the present disclosure;

FIG. 5 is a flow diagram of an exemplary method for GERD treatment, consistent with embodiments of the present disclosure;

FIG. 6 is a flow diagram of an exemplary method for obesity treatment, consistent with embodiments of the present disclosure;

FIG. 7 is a diagram showing strain changes sensed at different times by a plurality of sensor devices S1-S4;

FIG. 8 is a perspective view of an exemplary coil, consistent with embodiments of the present disclosure; and

FIG. 9 is a perspective view of an exemplary coil, consistent with embodiments of the present disclosure.

### DESCRIPTION OF THE EMBODIMENTS

Reference will now be made in detail to the exemplary embodiments of the disclosure, examples of which are illustrated in the accompanying drawings. Wherever possible, the same reference numbers will be used throughout the drawings to refer to the same or like parts.

In exemplary embodiments of the present disclosure, GERD and obesity treatments are performed using an alimentary tract treatment system including one or more sensor assemblies, a control unit, and one or more alimentary tract treatment devices. The treatments are initiated by monitoring locations along the alimentary tract using the one or more sensor assemblies. The sensor assemblies generate input signals indicative of sensed strain, or other suitable parameters, at the monitored locations. Next, the control unit receives the input signals and executes one or more algorithms used to determine whether the input signals are indicative of one or more digestive events. Based on the determination, the control unit may send a command signal to the one or more alimentary tract treatment devices. The one or more alimentary tract treatment devices may include one or more valve devices and/or one or more electric stimulation devices for controlling movement of material through the alimentary tract, thereby treating GERD and obesity. Though this description describes treatment devices for GERD and obesity, embodiments of the present disclosure may treat other ailments associated with the alimentary tract.

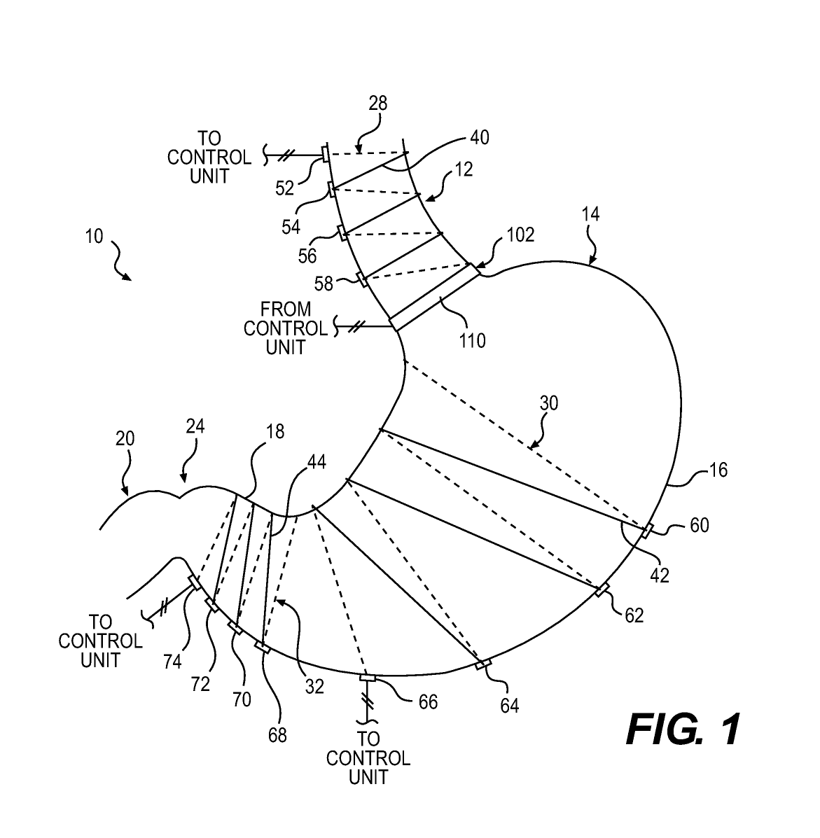

Exemplary features of the one or more sensor assemblies and the input signals they generate will now be described. FIG. 1 shows an illustration of a section of the alimentary tract 10 including a lower portion of an esophagus 12, a stomach 14, and an upper portion of a small intestine 20. One or more sensor assemblies 28-32 may be mounted on the alimentary tract section 10. The one or more sensor assemblies 28-32 may be put in place using laparoscopic surgical procedures or other suitable methods. The one or more sensor assemblies 28-32 may include one or more strain sensor assemblies, pH sensor assemblies, force sensor assemblies, and/or pressure sensor assemblies.

For example, pH sensor assemblies may be used to monitor pH values of alimentary tract fluids, including stomach fluids. pH monitoring may be performed continuously. One or more sensors forming a pH sensor assembly may be mounted on an inner surface of the alimentary tract 10, such as at inner surfaces of the esophagus 12, stomach 14, antrum 18, and/or pylorus 24.

Although strain sensor assemblies may be described in the exemplary embodiments below, it should be understood that any of the types of sensor assemblies may also be used.

The strain sensor assembly 28 may be supported by an outer surface of the esophagus 12 for the purpose of monitoring the esophagus 12. The strain sensor assembly 28 may include a coil 40 and one or more strain sensor devices 52-58. The coil 40 wraps around the esophagus 12 and is spring-biased to exert a compressive force on the esophagus 12, sufficient for keeping the coil 40 in place without interfering with normal esophagus operation. The coil 40 moves with and remains in contact with the outer surface of the esophagus 12 as the esophagus 12 expands and contracts. The strain sensor devices 52-58 are held against the outer surface of the esophagus 12 by the coil 40, and like the coil 40, the strain sensor devices 52-28 move with and remain in contact with the outer surface of the esophagus 12 as the esophagus 12 expands and contracts. Although the strain sensor devices 52-28 are shown as being arranged in a linear fashion on one portion of the outer surface of the esophagus 12 in FIG. 1, it is contemplated that two or more of the strain sensor devices 52-28 may be arranged on any other portion of the outer surface of the esophagus 12. It is further contemplated that the strain sensor devices 52-28 may be split up with one or more of the strain sensor devices resting on one portion of the outer surface of the esophagus 12, and one or more of the strain sensor devices 52-28 resting on another portion of the outer surface of the esophagus 12.

The strain sensor assembly 30 and the strain sensor assembly 32 may be mounted on the outer surface of the stomach 14 for the purpose of monitoring the stomach 14. The strain sensor assembly 30 may be supported by a main body 16 of the stomach 14. The strain sensor assembly 32 may be supported by an antrum 18 of the stomach 14.

The strain sensor assembly 30 includes a coil 42 that wraps around the main body 16. The coil 42 is similar to the coil 40 in structure and function. The spring-bias of the coil 42 allows it to move with and remain in contact with the outer surface of the main body 16 as the main body 16 expands and contracts. The strain sensor devices 60-66 are similar to the strain sensor devices 52-58 in structure and function. The strain sensor devices 60-66 are held against the outer surface of the main body 16 by the coil 42, and like the coil 42, the strain sensor devices 60-66 move with and remain in contact with the outer surface of the main body 16 as the main body 16 expands and contracts. While the strain sensor devices 60-66 are shown as being arranged linearly on one portion of the outer surface of the main body 16 in FIG. 1, it is contemplated that two or more of the strain sensor devices 60-66 may be arranged linearly on any other portion of the outer surface of the main body 16. It is further contemplated that the strain sensor devices 60-66 may be split up so one or more of the devices are on one portion of the outer surface of the main body 16, and one or more of the devices are on another portion of the outer surface of the main body 16.

The strain sensor assembly 32 includes a coil 44 that wraps around the antrum 18, and strain sensor devices 68-74 are held on the antrum 18 by the coil 44. The coil 44 and strain sensor devices 68-74 are similar to the coils 40 and 42 and the strain sensor devices 52-66 in structure and function. As such, the coil 44 and strain sensor devices 68-74 remain in contact with the outer surface of the antrum 18 as the antrum 18 expands and contracts. Two or more of the strain sensor devices 68-74 may be arranged linearly along any portion of the antrum 18. It is also contemplated that the strain sensor devices 68-74 may be split up with one or more of the devices on one portion of the outer surface of the antrum 18, and one or more of the devices on another portion of the outer surface of the antrum 18.

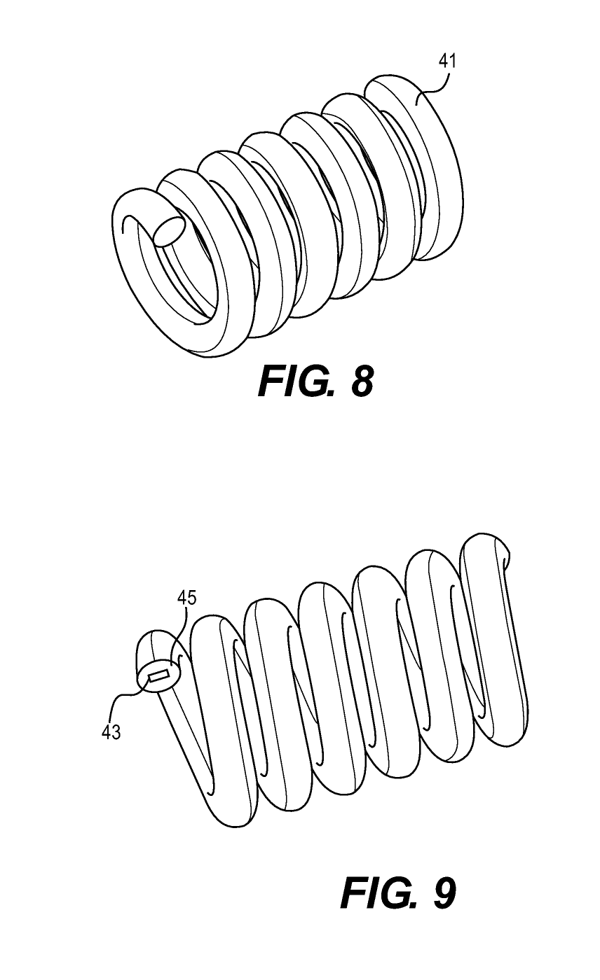

One or more of the coils 40, 42, and 44 may be formed by metal alloys including, for example, stainless steel and/or platinum chromium. These materials may have a desired amount of strength, and also radiopacity. The specific type of or composition of metal alloys may be selected such that movement or deformation of the material, by the alimentary tract 10, may not lead to permanent deformation of the material. It is also contemplated that one or more of the coils 40, 42, and 44 may be formed by biocompatible polymers. FIG. 8 shows a coil 41 formed of metal alloy or a biocompatible polymer.

One or more of the coils 40, 42, and 44 may be formed of an assembly including a metal core 43 covered with a silicone layer 45, as shown in FIG. 9. The metal core 43 may be thinner, stronger, and more radiopaque than the silicone layer 45. The metal for the metal core 43 may include, for example, stainless steel and/or platinum chromium. It is also contemplated that one or more of the coils 40, 42, and 44 may be formed of Nitinol, and/or other shape memory alloys, including alloys of platinum, such as platinum-iridium alloys. A shape memory alloy coil may be desirable for its ability to retain its shape even when subjected to deformation.

One or more of the coils 40, 42, and 44 may have a substantially circular cross-section, as shown in FIG. 8. Alternatively, one or more of the coils 40, 42, and 44 may have an elliptical cross-section, as shown in FIG. 9. It is contemplated that any other suitable cross-sectional shape may also be used.

Any of the coils 40, 42, and 44 may include one section with one characteristic or set of characteristics, and another section with another characteristic or set of characteristics that is different. For example, one section of the coil may be made of one of the above-described materials, while another section of the coil may be made of another of the above-described materials that is different. It is also contemplated that one section of the coil may have one thickness, while another section of the coil may have a different thickness. It is further contemplated that one section of the coil may have one cross-sectional shape, while another section of the coil may have a different cross-sectional shape.

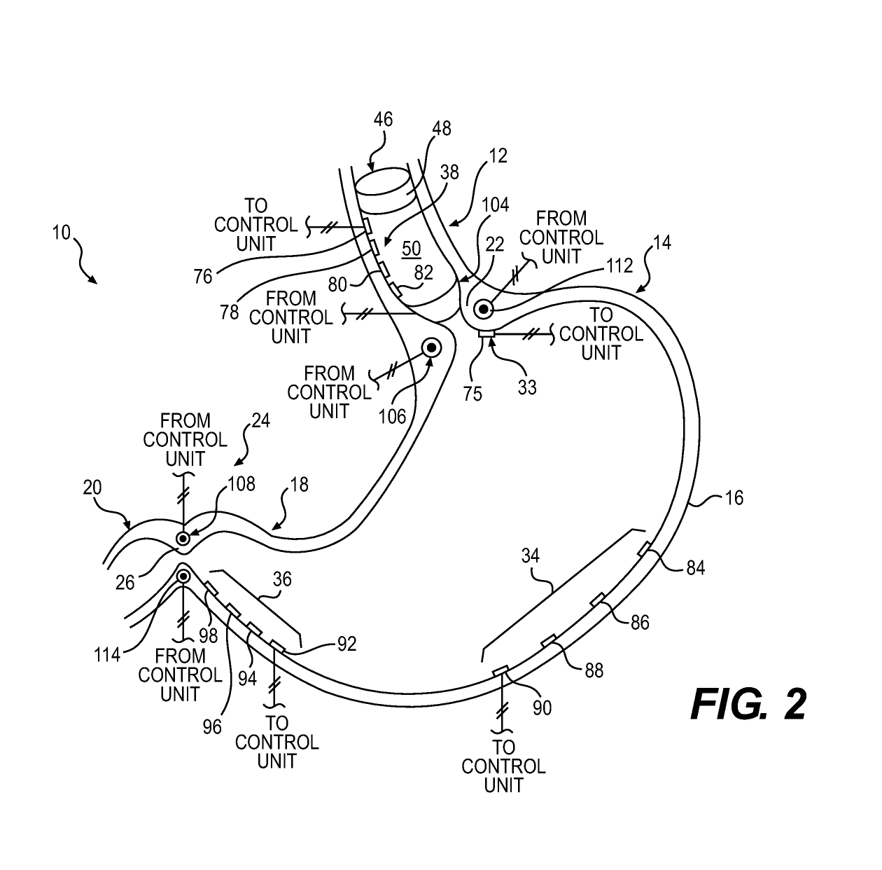

FIG. 2 is a cross-sectional illustration depicting interior regions of the alimentary tract section 10, and showing the locations of a lower esophageal sphincter (LES) 22 and a pyloric sphincter 26. One or more strain sensor assemblies 33-38 may be placed inside the alimentary tract section 10. The strain sensor assemblies 33-38 may be put in place using endoscopic surgical procedures or any other suitable method.

The strain sensor assemblies 33-36 may be supported by the inner surface of a wall of the stomach 14. For example, one or more of the strain sensor assemblies 33-36 may be implanted on or into the wall of the stomach 14 or otherwise attached to the inner surface of the stomach 14 using endoscopic surgical procedures.

The strain sensor assembly 33 may be placed at or near the junction of the main body 16 and the LES 22. The strain sensor assembly 33 may include a strain sensor device 75 configured to monitor gastric pressure at the junction. While the strain sensor device 75 is shown as being on one portion of the junction in FIG. 2, it is contemplated that the strain sensor device 75 may be mounted at any other portion of the junction.

The strain sensor assemblies 34 and 36 may each include one or more strain sensor devices 84-98 placed along the inner surface of the wall of the stomach 14. The strain sensor devices 84-90 may be placed along the main body 16 of the stomach 14. The strain sensor devices 92-98 may be placed along the antrum 18 of the stomach 14. The strain sensor devices 84-98 move with and remain in contact with the wall of the stomach 14 as the stomach 14 expands and contracts. While the strain sensor devices 84-90 are shown as being placed linearly along particular portions of the inner surface of the main body 16 in FIG. 2, it is contemplated that two or more of the strain sensor devices 84-90 may be placed linearly along any other portion of the inner surface of the main body 16. Further, two or more of the strain sensor devices 84-90 may be arranged nonlinearly along any portion of the inner surface of the main body 16. The same is true for placement of the strain sensor devices 92-98 along the inner surface of the antrum 18.

The strain sensor assembly 38 may be supported by a self-expandable stent 46 inserted within the esophagus 12. The strain sensor assembly 38 may be supported by a wall 48 of the stent 46, or by a cover 50 covering the stent 46. The expansion force generated by the stent 46 holds the strain sensor assembly 38 in contact with the inner surface of the esophagus 12, as the esophagus 12 expands and contracts. Two or more of the strain sensor devices 76-82 can be arranged substantially linearly along a portion of the stent 46 and the inner surface of the esophagus 12. It is also contemplated that two or more of the strain sensor devices 76-82 can be arranged around the stent 46 and the inner surface of the esophagus 12 in a substantially nonlinear manner.

The strain sensor devices 52-98 may include one or more fiber Bragg grating (FBG) sensors. A FBG sensor includes a fiber optic cable (not shown) having a central core (not shown) that has a refractory index that is higher than that of a shell (not shown) surrounding the central core. This enables the central core to transmit light via internal reflection. A FBG is generated by creating notches (not shown) within the central core. Light is reflected at specific frequencies as it hits the discontinuities at the FBG. The frequency of the reflected light is a function of strain on the fiber optic cable at the location of the FBG. Thus, the change in frequency can be used to sense a change in strain along the section of the fiber optic cable at the location of the FBG.

It is also contemplated that the strain sensor devices 52-98 may include one or more strain gauges. One exemplary type of strain gauge may be applied to a surface, and may be configured to deform when the surface deforms. Deformation of the strain gauge may produce a change in its electrical resistance. The change in electrical resistance may be monitored, and may be indicative of strain at the surface.

It is further contemplated that one or more of the sensor devices 52-98 may not sense strain or changes in strain, but rather, may sense pH, pressure, and/or force, or changes in pH, pressure, and/or force. While strain forms the basis for generating signals indicative of conditions in the alimentary tract 10 in the exemplary embodiments below, it should be understood that pH, pressure, and/or force may be used to generate similar signals.

Movement of the monitored areas of the alimentary tract section 10, and/or pressure changes in the monitored areas of the alimentary tract section 10, may change the level of strain on the strain sensor devices 52-98. The strain sensor devices 52-98 will generate one or more input signals indicative of strain in the areas in which they are mounted. By comparing strain signals over time, changes in strain at the strain sensor devices can be identified. The magnitudes of any sensed strain changes, the relative timing of any sensed strain changes, and/or the locations of the strain sensor devices 52-98 reporting strain changes may indicate that one or more digestion events (e.g., swallowing, stomach contraction, and vomiting) have occurred.

The strain sensor assemblies 28-38 and their corresponding strain sensor devices 52-98 may be operatively coupled to a control unit (not shown) to provide strain signals to the control unit. The control unit may be implantable or carried on the body. The strain sensor assemblies 28-38 may be coupled to the control unit by wires including, for example, wires extending from one or more of the strain sensor devices 52-98 to the control unit. It is also contemplated that the strain sensor assemblies 28-38 and one or more of the strain sensor devices 52-98 may communicate with the control unit wirelessly via a wireless communications medium.

The control unit may include an input element (not shown) configured to receive the strain signals. Any one of or combination of the strain sensor assemblies 28-38 and their strain sensor devices 52-98 shown in FIGS. 1 and 2 may be used to supply input signals to the control unit through, for example, wires or a wireless communications medium.

The control unit may also include a memory element (not shown) configured to store the strain signals and other calculated values. For example, the memory element may include threshold pressure/strain amounts, for comparing to the received strain signals, to determine if one or more digestions events are occurring. Additionally or alternatively, the memory element may include threshold pH, pressure, and/or force amounts, for comparing to received pH, pressure, and/or force signals, to determine if one or more digestion events are occurring.

Threshold amounts or values may be preprogrammed into the memory element, and/or may be determined using one or more of the sensor devices 52-98. For example, with respect to the esophagus 12, one or more of the sensors at or on the esophagus 12 may be used to take baseline measurements when a subject is not eating. For example, baseline measurements may be taken at night. One or more thresholds may be set relative to the baseline measurements. For example, thresholds may be set at five standard deviations from the baseline measurements.

The control unit may also include a processor element (not shown) configured to apply one or more algorithms to the received signals from the sensor devices 52-98, to determine when a digestion event has occurred in the esophagus 12. For example, in one exemplary algorithm, whenever the control unit receives an indication that a plurality of the sensor devices at or on the esophagus 12 have crossed their thresholds, the control unit may evaluate the temporal sequence with which the thresholds were crossed.

If the temporal sequence of crossing moves in a downstream direction of the esophagus 12, the control unit may determine that swallowing is occurring. If the temporal sequence moves in an upstream direction of the esophagus, the control unit may determine that acid reflux or vomiting is occurring.

With respect to the stomach 14, one or more of the sensors at or on the stomach 14 may be used to take baseline measurements during normal gastric digestion. During normal gastric digestion a cyclical pattern of sensed values may be expected. The control unit may use one or more aspects of the cyclical pattern as a reference for normal gastric digestion. For example, during normal gastric digestion cycles, there may be a temporal sequence of strain peaks across sensors from the antrum 18 to the pylorus 24. The control unit may use the strain peak levels and/or timing as a reference or baseline. Strain during a gastric event like vomiting may be greater than strain during normal gastric digestion. Also, during vomiting, strain peaks may occur closer in time, for example almost simultaneously, across multiple sensors, rather than sequentially or farther apart in time as in normal gastric digestion. Thus, using the strain peaks for normal gastric digestion as a baseline, one or more threshold values for strain peak levels and/or strain peak timing may be set that, when crossed, may be indicative of vomiting.

The control unit may also include an output element (not shown) configured to generate a command signal to activate or deactivate at least one of a laparoscopic valve device 102, an endoscopic valve device 104, and electrical stimulation devices 106 and 108 based on the strain signals and/or determinations of digestion event occurrences. It is contemplated that the output element may be in operative communication with one or more of the valve device 102, the valve device 104, the electrical stimulation device 106, and the electrical stimulation device 108, through connecting wires or a wireless communications medium, through which the command signals are transmitted.

It is also contemplated that the control unit may include a power source (not shown) for powering the strain sensor assemblies 28-38, valve devices 102 and 104, and electric stimulation devices 106 and 108.

The laparoscopic valve device 102 is shown in FIG. 1. The valve device 102 may include a band or ring member 110 placed around the esophagus 12 on a region surrounding the LES 22. The band 110 may be selectively actuated between contracted and expanded positions based on command signals from the control unit. Contraction of the band 110 assists closing of the LES 22, thus reducing or preventing material flow through the LES 22. Expansion of the band 110 permits easier opening of the LES 22, allowing greater material flow through the LES 22. The generating of command signals controlling the timing of contracting and expanding of the band 110, and/or the force of contraction and expansion of the band 100, may be based on the type of strain signals received and/or user defined instructions programmed into the control unit.

The endoscopic valve device 104 is shown in FIG. 2. The valve device 104 may be mounted on an end of the stent 46, and particularly the distal end closest to the LES 22. The valve device 104 may be selectively actuated to move between a flow restricting position for reducing or preventing material flow through the LES 22, and a flow permitting position for increasing material flow through the LES 22. The generating of command signals controlling the timing of opening and closing of the valve device 104 may be based on the type of strain signals received and/or user defined instructions programmed into the control unit.

The electric stimulation devices 106 and 108 are shown in FIG. 2. The electric stimulation device 106 may include one or more electrodes 112 implanted on or in tissue at or around the LES 22. The electric stimulation device 108 may include one or more electrodes 114 implanted on or in tissue at or around the pyloric sphincter 26. The number, size, and arrangement of electrodes at a sphincter may be based on the sphincter's size, shape, tissue composition, and/or on the amount of electric stimulation desired. Activation of one or more electrodes at a sphincter sends electricity into the sphincter tissue, causing contraction of sphincter, thereby assisting with closing of the sphincter. Deactivation of the electrodes removes the electric stimulus. The generating of command signals controlling the timing of electrical stimulation, and/or the properties of the electric stimulation provided (e.g., voltage, current, frequency, and duration), may be based on the type of strain signals received and/or user defined instructions programmed into the control unit.

Methods for GERD and/or obesity treatment using at least one of the sensor assemblies 28-38, the control unit, and at least one of the valve devices 102 and 104 and electric stimulation devices 106 and 108, will now be described.

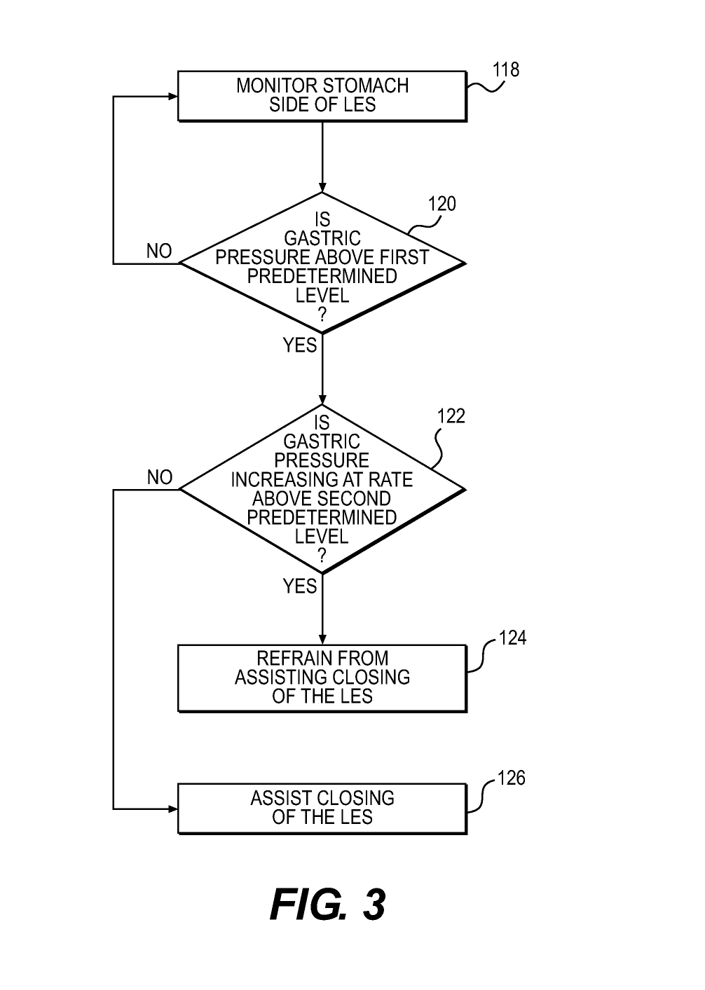

The steps 118-126 of one exemplary method for GERD treatment are shown in FIG. 3. The step 118 includes monitoring a stomach side of the LES 22 at or near the junction of the esophagus 12 and stomach 14. The strain sensor assembly 33 can be used for monitoring, and may provide strain signals to the control unit that are indicative of gastric pressure near the junction. The control unit may run one or more algorithms using the strain signals as inputs. Using the algorithms, the control unit can determine whether the strain signals, and thus the gastric pressure, are above a first predetermined value (step 120). If the control unit determines that the gastric pressure is not above a first predetermined level (“NO”), monitoring will continue. If the control unit determines that the gastric pressure is above the first predetermined level (“YES”), the control unit may then determine whether the gastric pressure is increasing at a rate that is above a second predetermined level (step 122). If the rate of increase is above the second predetermined level (“YES”), the control unit may interpret the intense rise in gastric pressure as being indicative of vomiting.

Additionally or alternatively, the control unit may recognize vomiting if readings from the sensors on or at the esophagus 12 cross thresholds, according to a temporal sequence, along an upstream direction of the esophagus 12. It is also contemplated that the control unit may recognize vomiting if one or more of the sensors at or on the stomach 14 experiences a strain peak that exceeds a threshold, and/or experience strain peaks occurring closer in time than would be the case during normal gastric digestion.

When vomiting is recognized by the control unit, the control unit may refrain from assisting closing of the LES 22 (step 124) so that vomiting is not hindered. If the rate of increase is not above the second predetermined level (“NO”), the control unit may interpret the rise in gastric pressure as being a precursor to or indicative of unwanted gastric backflow typical of GERD. Accordingly, the control unit may assist closing of the LES 22 (step 126) to reduce or prevent the backflow.

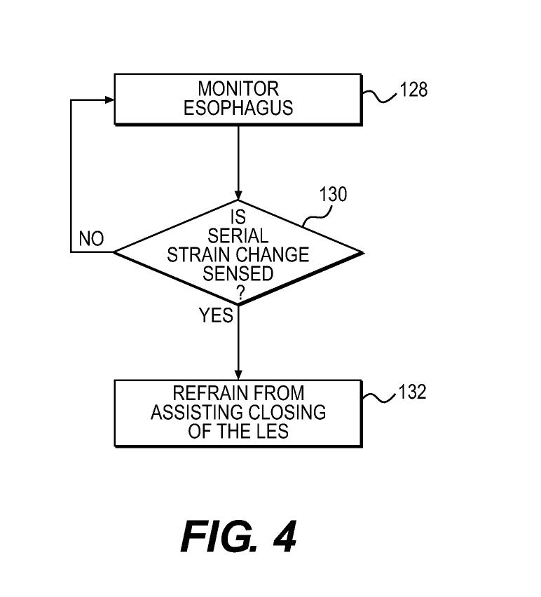

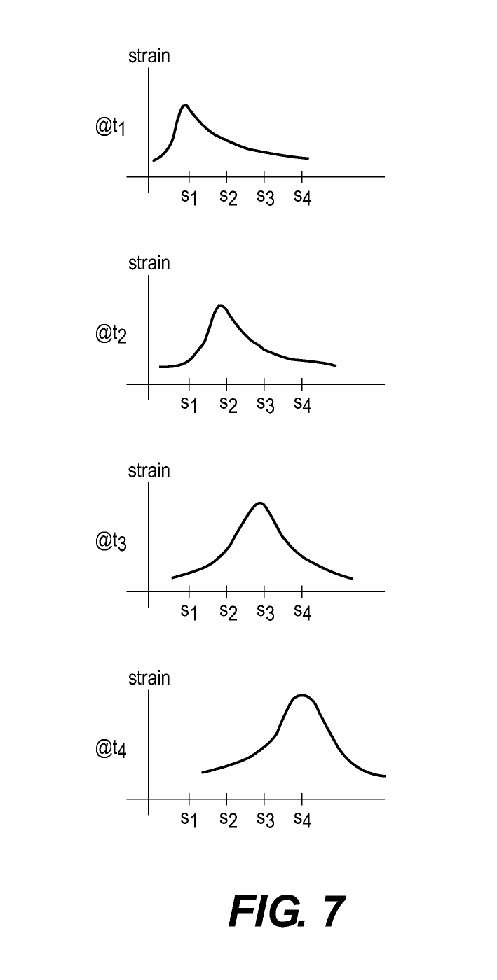

The steps 128-132 of another exemplary method for GERD therapy are shown in FIG. 4. The step 128 includes monitoring the esophagus 12. The sensor assembly 28 and/or the sensor assembly 38 can be used for monitoring the esophagus 12 and generating strain signals indicative of changes in movement and/or pressure of the esophagus 12. The control unit can receive the strain signals and determine whether a serial strain change, similar to the serial strain change depiction in FIG. 7, has occurred (step 130). For example, if the strain signals sent from the strain sensor devices 52-58 and/or the strain sensor devices 76-82 to the control unit resemble the strain signals from sensor devices S1-S4 in FIG. 7, in terms of magnitude and timing (at times t1-t4), a serial strain change has occurred. This may be caused by a peristaltic wave travelling through the alimentary tract section 10, and in this instance, through the esophagus 12 during swallowing. If a serial strain change has occurred (“YES”), the control unit may interpret the occurrence as being indicative of swallowing. As such, the control unit may refrain from assisting with closing of the LES 22 (step 132) so that swallowing is not hindered. If a serial strain change has not occurred (“NO”), the control unit may continue monitoring the esophagus 12.

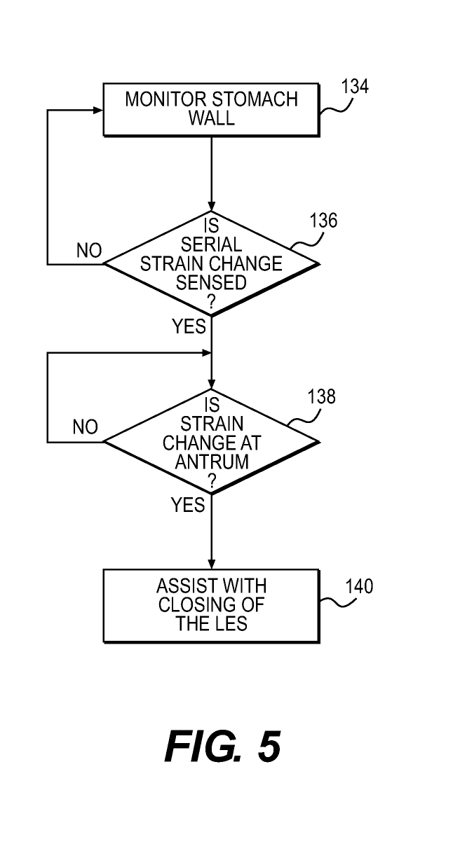

The steps 134-140 of another exemplary method for GERD therapy are shown in FIG. 5. The step 134 includes monitoring the wall of the stomach 14. Sensor assembly 30 and/or sensor assembly 34 can be used for monitoring the wall of the stomach 14 and generating strain signals indicative of changes in movement and/or pressure of the wall of the stomach. The control unit can receive the strain signals and determine whether a serial strain change for the strain sensor devices 60-66 and/or strain sensor devices 84-90, similar to the serial strain change depiction for sensors S1-S4 in FIG. 7, has occurred (step 136). The serial strain change may be caused by a peristaltic wave travelling through the stomach 14. If such a serial strain change has not occurred (“NO”), no action will be taken and monitoring will continue. If such a serial strain change has occurred (“YES”), the control unit may determine whether the serial strain change, and thus the peristaltic wave, has reached the antrum 18 (step 138). For example, if a sensor device in the antrum 18 forms part of the serial strain change, that indicates that the peristaltic wave and resultant serial strain change has reached the antrum 18. If this is the case (“YES”), the control unit may assist with closing of the LES 22 to prevent gastric backflow (step 140). If the serial strain change has not reached the antrum 18 (“NO”), the control unit will wait until it has occurred before taking any action. By waiting for the serial strain change to reach the antrum 18, the control unit can avoid hindering swallowing. Also, since the control unit only assists with closing of the LES 22 during a specified time period, battery life can be conserved.

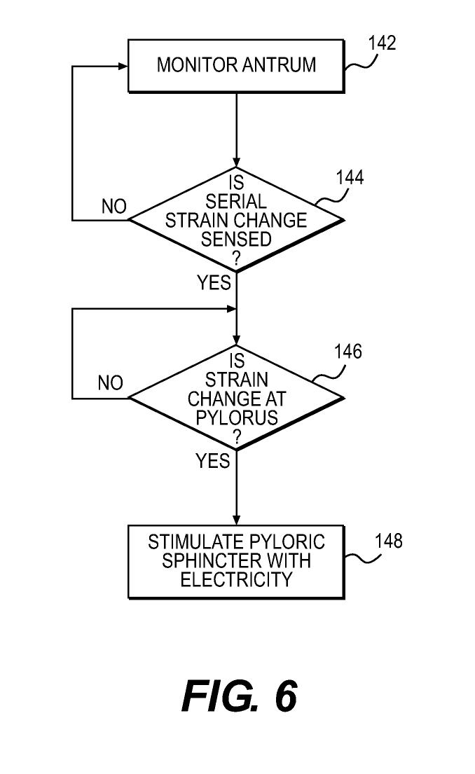

The steps 142-148 of an exemplary method for obesity treatment are shown in FIG. 6. The step 142 includes monitoring the antrum 18. Sensor assembly 32 and/or sensor assembly 36 can be used for monitoring the antrum 18 and generating strain signals indicative of changes in movement and/or pressure of the antrum 18. The control unit can receive the strain signals and determine whether a serial strain change for the strain sensor devices 68-74, similar to the serial strain change for the sensors S1-S4 depiction in FIG. 7, has occurred (step 144). The serial strain change may be caused by a peristaltic wave travelling through the antrum 18. If such a serial strain change has not occurred (“NO”), no action will be taken and monitoring will continue. If such a serial strain change has occurred (“YES”), the control unit may determine whether the serial strain change, and thus the peristaltic wave, has reached the pylorus 24 of the stomach 14 (step 146). For example, if a sensor device close to or at the pylorus 24 forms part of the serial strain change, that indicates that the peristaltic wave and resultant serial strain change has reached the pylorus 24. If this is the case (“YES”), the control unit may assist with closing of the pyloric sphincter 26 to reduce or prevent material flow from the stomach 14 into the small intestine 20 (step 148). This may extend the feeling of satiety experienced by the patient. If the serial strain change has not reached the pylorus 24 (“NO”), the control unit will wait until it has reached it before taking action. By waiting for the serial strain change to reach the pylorus 24, the control unit can maximize the efficacy of the system while preserving battery life.

In the exemplary methods for GERD treatment described above and shown in FIGS. 3-5, the step of refraining from assisting closing of the LES 22 may include triggering or otherwise allowing expansion of the valve device 102, opening the valve device 104, and/or deactivating the electric stimulation device 106. Conversely, the step of assisting closing of the LES 22 may include triggering or otherwise allowing contraction of the valve device 102, closing the valve device 104, and/or activating the electric stimulation device 106. It is contemplated that the methods for GERD treatment may be performed individually, in any combination of two or more, in series, and/or simultaneously.

In the exemplary method for obesity treatment described above and shown in FIG. 6, the step of refraining from assisting closing of the pyloric sphincter 26 may include deactivating the electric stimulation device 108. The step of assisting closing of the pyloric sphincter 26 may include activating the electric stimulation device 108. It is contemplated that the method for obesity treatment may be performed individually or in combination with one or more of the methods for GERD treatment shown in FIGS. 3-5. When performed in combination, the method steps may be performed serially or simultaneously.

Other embodiments of the present disclosure will be apparent to those skilled in the art from consideration of the specification and practice of the embodiments disclosed herein. It is intended that the specification and examples be considered as exemplary only, with a true scope and spirit of the present disclosure being indicated by the following claims.

What is claimed is:

1. An alimentary tract treatment system, comprising:
a plurality of sensor devices for engaging a wall of a portion of the alimentary tract, and for sensing a parameter of the wall; and
an alimentary tract treatment device for controlling a flow of material through the alimentary tract, wherein operation of the alimentary tract treatment device is controlled based on the parameter sensed by the plurality of sensor devices.

2. The alimentary tract treatment system of claim 1, wherein the plurality of sensor devices are serially arranged strain sensor devices, and the parameter is peristaltic movement of the wall.

3. The alimentary tract treatment system of claim 2, further including at least one tissue engaging portion for connecting the plurality of sensor devices and engaging tissue of the wall.

4. The alimentary tract treatment system of claim 3, wherein the at least one tissue engaging portion includes a coil for wrapping at least partially around and engaging outer tissue of the wall.

5. The alimentary tract treatment system of claim 4, wherein the coil is biased for maintaining engagement between the coil and the outer tissue of the wall during movement of the wall.

6. The alimentary tract treatment system of claim 4, wherein the plurality of serially arranged strain sensor devices are coupled to the coil for mounting the plurality of serially arranged strain sensor devices on the outer tissue of the wall.

7. The alimentary tract treatment system of claim 3, wherein the at least one tissue engaging portion includes a self-expandable stent for engaging inner tissue of the wall, and the plurality of serially arranged strain sensor devices are coupled to the stent for mounting the plurality of serially arranged strain sensor devices on the inner tissue of the wall.

8. The alimentary tract treatment system of claim 1, wherein the alimentary tract treatment device includes at least one of a valve and at least one electrical stimulation device for stimulating tissue.

9. The alimentary tract treatment system of claim 8, wherein the valve includes a circular band with an adjustable opening extending therethrough.

10. The alimentary tract treatment system of claim 2, wherein the plurality of serially arranged strain sensor devices include one or more fiber Bragg grating sensors.

11. The alimentary tract treatment system of claim 2, wherein the plurality of serially arranged strain sensor devices are operatively coupled to the alimentary tract treatment device by a control unit.

12. The alimentary tract treatment system of claim 1, wherein the control unit receives input signals from the plurality of sensor devices and transmits output signals to the treatment device.

13. The alimentary tract treatment system of claim 1, wherein the plurality of sensor devices include at least one of a pH sensor, a pressure sensor, and a strain gauge.

14. A method for treating gastroesophageal reflux disease (GERD), comprising:
sensing peristaltic movement of a portion of an alimentary tract using a plurality of serially arranged strain sensor devices, wherein the plurality of serially arranged strain sensor devices are mounted on a wall of the alimentary tract, and are operatively coupled to a GERD treatment device at a region of the alimentary tract including a lower esophageal sphincter (LES); and
controlling operation of the GERD treatment device based on the sensed peristaltic movement.

15. The method for treating GERD of claim 14, wherein the GERD treatment device includes at least one of a valve and an electrical stimulation device.

16. The method for treating GERD of claim 14, wherein controlling operation of the GERD treatment device includes operating at least one of the valve and the electrical stimulation device in a first state assisting closing of the LES.

17. The method for treating GERD of claim 14, wherein controlling operation of the GERD treatment device includes operating at least one of the valve and the electrical stimulation device in a second state refraining from assisting closing of the LES.

18. A method for treating obesity, comprising:
sensing a peristaltic movement of a portion of an alimentary tract using a plurality of serially arranged sensor devices, wherein the plurality of serially arranged sensor devices are mounted on a wall of the alimentary tract, and are operatively coupled to an obesity treatment device at a pyloric region of the alimentary tract; and
controlling operation of the obesity treatment device based on the sensed peristaltic movement.

19. The method for treating obesity of claim 18, wherein controlling operation includes triggering electrical stimulation of pyloric region tissue based on the sensed peristaltic movement.

20. The method for treating obesity of claim 18, wherein controlling operation includes triggering electrical stimulation of pyloric region tissue based on the sensed peristaltic movement reaching a predetermined one of the plurality of serially arranged strain sensor devices.
